SEQUENCE LISTING (1 sequence)

SEQ ID NO: 1 - DNA, 2767 bp, Artificial Sequence
  Synthetic construct
ggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggccatggcacacccttaggtaacccagtagatccagaggaattcattatcagtgcaattgttttgtcacgatcaaaggactctggtacaaaatcgtattcattaaaaccgggaggtagatgagatgtgacgaacgtgtacatcgactgaaatccctggtaatccgttttagaatccatgataataattttctggattattggtaattttttttgcacgttcaaaattttttgcaacccctttttggaaacaaacactacggtaggctgcgaaatgttcatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtattccgcgtacgtgatgttcacctcgatatgtgcatctgtaaaagcaattgttccaggaaccagggcgtatctcttcatagccttatgcagttgctctccagcggttccatcctctagaggatagaatggcgccgggcctttctttatgtttttggcgtcttcacgcgtcgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtgggatccaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

(19) United States
(12) Patent Application Publication
(10) Pub. No.: US 2006/0026711 A1
(43) Pub. Date: Feb. 2, 2006

(54) Non-systemic gene suppression in plants

(76) Inventors: Shihshieh Huang, Stonington, CT (US); Larry A. Gilbertson, Chesterfield, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 11/186,196

(22) Filed: Jul. 20, 2005

Related U.S. Application Data
(60) Provisional application No. 60/589,643, filed on Jul. 21, 2004.

(51) Int. Cl. A01H 1/00; C12N 15/82

(52) U.S. Cl. 800/279; 435/468

(57) ABSTRACT

The present invention provides methods for non-systemic gene suppression of target gene or a plant of a plant pest or pathogen. The invention further provides transgenic plants having non-systemic resistance to a pest or pathogen.

This application claims the benefit of priority of U. S. Provisional Patent Application 60/589,643, which was filed on 21 Jul. 2004 and is incorporated by reference in its entirety herein. The sequence listing contained in the file named “38-21(53709)C.rpt”, which is 4 kilobytes (measured in MS-Windows) and located in computer readable form on a compact disk created on 20 Jul. 2005, is filed herewith and incorporated herein by reference

### FIELD OF THE INVENTION

The present invention discloses molecular constructs and methods for non-systemic gene suppression in plants, for example, non-systemic suppression of a target gene of a plant or of a plant pest or pathogen. Also disclosed are transgenic plants and seeds whose genome includes molecular constructs for non-systemic gene suppression.

### BACKGROUND OF THE INVENTION

Anti-sense gene suppression in plants is described by Shewmaker et al. in U.S. Pat. Nos. 5,107,065; 5453,566; and 5,759,829.

Gene suppression using which is complementary to mRNA is disclosed by Inouye et al. in U.S. Pat. Nos. 5,190,931; 5,208,149 and 5,272,065.

Carmichael et al. in U.S. Pat. Nos. 5,908,779 and 6,265,167 discloses methods and constructs for expressing and accumulating anti-sense RNA in the nucleus using a construct that comprises a promoter, anti-sense sequences, and sequences encoding a cis-or trans-ribozyme. The cis-ribozyme is incorporated into the anti-sense construct in order to generate 3′-ends independently of the polyadenylation machinery and thereby inhibit transport of the RNA molecule to the cytoplasm. Carmichael demonstrated the use of the construct in mouse NIH 3T3 cells.

The efficiency of anti-sense gene suppression is typically low. Redenbaugh et al. in “Safety Assessment of Genetically Engineered Fruits and Vegetables”, CRC Press, 1992 report a transformation efficiency ranging from 1% to 20% (page 113) for tomato transformed with a construct designed for anti-sense suppression of the polygalacturonase gene. Chuang et al. reported in PNAS (2000) 97:9, 4985-4990 that anti-sense constructs, sense constructs and constructs where anti-sense and sense DNA are driven by separate promoters had either no, or weak, genetic interference effects as compared to potent and specific genetic interference effects from dsRNA constructs (see FIG. 1 and Table 1). See also Wesley et al. who report in The Plant Journal (2001) 27(6), 581-590, e. g. at Table 1, the comparative efficiency of hairpin RNA, sense and anti-sense constructs at silencing a range of genes in a range of plant species with a clear indication that the efficiency for anti-sense constructs is typically about an order of magnitude lower than the efficiency for hairpin RNA.

Matzke et al. in Chapter 3 “Regulation of the Genome by double-stranded RNA” of RNAi—A guide to Gene Silencing, Cold Spring Harbor Laboratory Press, Edited by Hannon, (2003), discuss the use of polyadenylation signals in promoter inverted repeat constructs. At page 58, they state “the issue of whether to put polyadenylation signals in promoter inverted repeat constructs is unsettled because the nature of the RNA triggering RdDM [RNA-directed DNA methylation] is unresolved. Depending on whether short RNA or dsRNA is involved in RdDM, the decision to include a polyadenylation site might differ depending on the experimental system used. If dsRNA is involved in RdDM, then a polyadenylation signal is not required because dsRNA forms rapidly by intramolecular folding when the entire inverted repeat is transcribed. Indeed, nonpolyadenylated dsRNAs might be retained in the nucleus and induce RdDM more efficiently than polyadenylated dsRNAs.

Matzke et al. continue: “If short RNAs guide homologous DNA methylation, then the situation in plants and mammals differ. In plants, which probably possess a nuclear form of Dicer, non-polyadenylated dsRNAs would still be optimal because they should feed preferentially into a nuclear pathway for dsRNA processing.”

### SUMMARY OF THE INVENTION

This invention provides methods and constructs for non-systemic gene suppression in plants. One aspect of this invention provides a method of non-systemic suppression of at least one target gene, including transcribing in a transgenic plant a recombinant DNA construct consisting of a promoter operably linked to a gene suppression element, wherein transcription of the gene suppression element produces RNA that non-systemically suppresses at least one target gene and is retained in the nucleus, thereby suppressing the target gene relative to expression in the absence of the transcription. Another aspect of this invention provides a method of tissue-specific control of a pest or pathogen of a plant, including providing a transgenic plant having in its genome a recombinant DNA construct including a tissue-specific promoter operably linked to a gene suppression element, wherein the gene suppression element transcribes to RNA that suppresses at least one gene of said pest or pathogen and is retained in the nucleus, thereby providing tissue-specific control of the pest or pathogen. Yet another aspect of this invention is a transgenic plant that is resistant to a pest or pathogen of the plant, wherein the transgenic plant has in its genome a recombinant DNA construct including a tissue-specific promoter operably linked to a gene suppression element, wherein the gene suppression element transcribes to RNA that suppresses at least one gene of the pest or pathogen and is retained in the nucleus, thereby providing tissue-specific control of the pest or pathogen. Transgenic plants of the invention include transformed plants, transgenic seeds, and transgenic plants grown from transgenic seeds.

In one embodiment of this invention anti-sense gene suppression in plants is enhanced by using a DNA construct comprising DNA for transcribing anti-sense RNA without DNA for transcribing a polyadenylation signal. In another embodiment of this invention anti-sense gene suppression is enhanced by using a DNA construct comprising DNA for transcribing anti-sense RNA without DNA for transcribing a polyadenylation signal and ribozymes; the transcribed anti-sense RNA is without a polyA tail or a ribozyme or other element providing double-strandedness. In yet another embodiment of this invention, non-systemic gene suppression is provided by using a DNA construct comprising DNA for transcribing RNA without DNA for transcribing a polyadenylation signal and ribozymes; the transcribed RNA is at least partially double-stranded RNA without a polyA tail or a ribozyme.

More specifically, constructs for non-systemic gene suppression in plants can include a promoter functional in plants operably linked to anti-sense oriented DNA for transcribing anti-sense RNA which is complementary to at least a segment of mRNA which is natively transcribed from a gene targeted for silencing. In another embodiment, constructs for non-systemic gene suppression in plants can include a promoter functional in plants operably linked to anti-sense and sense oriented DNA for transcribing anti-sense RNA which is complementary to at least a segment of mRNA which is natively transcribed from a gene targeted for silencing the construct and sense RNA which is complementary to the anti-sense RNA. The transcribed RNA can comprise at least 20 to upwards of 1000 or more nucleotides. More specifically, short anti-sense RNA can comprise 20 to 27 nucleotides in length, e.g. 21 or 23 nucleotides. Longer anti-sense RNA can comprise 30 to 1000 nucleotides, e.g. about 100 or 300 nucleotides. More particularly, the anti-sense oriented DNA can be chimeric, e.g. comprising a fusion of DNA from a plurality of genes targeted for suppression, or multiple copies of one or more anti-sense DNA sequences.

The plant functional promoter in such constructs can be selected depending on the nature of the intended gene silencing. For ubiquitous gene silencing a constitutive, ubiquitous promoter such as a CaMV35S promoter can be used. For tissue specific gene silencing, e.g. in roots or seed, a root specific promoter or a seed specific promoter can be used. For condition-induced gene silencing, e.g. water-deficit, a water deficit-inducible promoter can be used.

The DNA construct can comprise certain 3′UTR DNA provided that polyadenylation signals or other elements that assist in RNA transfer into the cytoplasm are not employed. In preferred aspects of this invention, the DNA constructs do not comprise any ribozyme elements or other elements that add double-stranded RNA segments to the transcribed anti-sense RNA.

In another embodiment of this invention enhanced anti-sense constructs are used to effect non-systemic, tissue specific gene silencing. Such constructs are useful for limiting gene suppression to specific tissue such as seeds or roots in plants. For instance, such enhanced anti-sense constructs can be used to modify the composition of oil, protein, starch or amino acid content of plant seeds by suppressing enzymes in biosynthetic pathways for such components. For example, transgenic maize having recombinant DNA for suppressing lysine ketoglutarate reductase (LKR) can be produced using an enhanced anti-sense construct consisting of a seed specific promoter operably linked to an anti-sense oriented DNA form a gene encoding LKR. Seed from such a transgenic maize plant with recombinant DNA having the enhanced anti-sense construct will have increased lysine as compared to seed of substantially equivalent genotype without the recombinant DNA.

One broader aspect of the invention provides a transgenic plant that is resistant to a pest or pathogen of the plant (e. g., a virus, bacterium, fungus, or invertebrate pest or pathogen), wherein the transgenic plant has in its genome a recombinant DNA construct comprising a tissue-specific promoter operably linked to a gene suppression element, wherein said gene suppression element transcribes to RNA that suppresses at least one gene of the pest or pathogen and is retained in the nucleus, thereby providing tissue-specific control of the pest or pathogen. In one embodiment, the transgenic plant has recombinant DNA for suppressing expression of protein from a native gene where the recombinant DNA consists of a promoter segment operably linked to an anti-sense DNA segment from the gene targeted for suppression. In a particularly preferred embodiment, the transgenic plant is a transgenic crop plant (such as, but not limited to, maize, rice, wheat, cotton, canola, and soybean) and the pest or pathogen can be an invertebrate (especially a pest insect or a pest nematode).

Other specific embodiments of the invention are disclosed in the following detailed description.

### BRIEF DESCRIPTION OF THE DRAWINGS

FIG. 1 schematically illustrates DNA vectors as described in Example 1. Legend: pale grey regions labelled “e35S-Hsp70”: a chimeric promoter element including an enhanced CaMV35S promoter linked to an enhancer element (an intron from heat shock protein 70 of Zea mays, Pe35S-Hsp70 intron); medium grey regions labeled “LUC”: DNA coding for firefly luciferase; dark grey regions labeled “3′ nos”: a 3′UTR DNA from Agrobacterium tumefaciens nopaline synthase gene. Vectors are conventionally depicted as transcribing from left (5′) to right (3′). Arrows indicate orientation of the luciferase segments as sense (arrowhead to right) or anti-sense (arrowhead to left).

FIG. 2 is a schematic map of a plasmid including an enhanced anti-sense construct as described in Example 6.

FIG. 3 is a schematic map of a vector including an enhanced anti-sense construct and described in Example 7. The plasmid includes an aroA gene as an herbicidal selectable marker, and a recombinant DNA construct for enhanced anti-sense gene suppression, consisting of a seed-specific maize L3 oleosin promoter operably linked to transcribable DNA consisting of about 300 base pairs of a maize lysine ketoglutarate reductase (LKR) gene (LKR region of the lysine ketoglutarate reductase//saccharopine dehydrogenase gene, LKR/SDH) in an anti-sense orientation, wherein a functional polyadenylation site is absent in this transcribable DNA, and left T-DNA border (LB) and right T-DNA border (RB) elements. An alterative vector contains an additional sense DNA sequence that is complementary to the LKR anti-sense sequence, allowing transcription of an at least partially double-stranded RNA from the construct.

FIG. 4 is a schematic map of a vector including an enhanced anti-sense construct and described in Example 8. The vector includes an aroA gene as an herbicidal selectable marker and a recombinant DNA construct for enhanced anti-sense gene suppression, consisting of a TUB-1 root specific promoter from Arabidopsis thaliana operably linked to transcribable DNA consisting of anti-sense oriented DNA of a nematode major sperm protein (msp) of a soybean cyst nematode, wherein a functional polyadenylation site is absent in this transcribable DNA. The plasmid also includes left T-DNA border (LB) and right T-DNA border (RB) elements. An alterative vector contains an additional sense DNA sequence that is complementary to the msp anti-sense sequence, allowing transcription of an at least partially double-stranded RNA from the construct.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Where a term is provided in the singular, the inventors also contemplate aspects of the invention described by the plural of that term. The nomenclature used herein and the laboratory procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries. The inventors do not intend to be limited to a mechanism or mode of action. Reference thereto is provided for illustrative purposes only.

A useful technology for building DNA constructs and vectors for transformation is disclosed in U.S. patent application Publication 2004/0115642 A1, incorporated herein by reference. Alternatively, DNA constructs can be built using the GATEWAY™ cloning technology (available from Invitrogen Life Technologies, Carlsbad, Calif.) which uses the site specific recombinase LR cloning reaction of the Integrase/att system from bacterophage lambda vector construction, instead of restriction endonucleases and ligases. The LR cloning reaction is disclosed in U.S. Pat. Nos. 5,888,732 and 6,277,608, U.S. patent application Publications 2001283529, 2001282319 and 20020007051, all of which are incorporated herein by reference. The GATEWAY™ Cloning Technology Instruction Manual which is also supplied by Invitrogen also provides concise directions for routine cloning of any desired DNA into a vector comprising operable plant expression elements. An alternative vector fabrication method employs ligation-independent cloning as disclosed by Aslanidis, C. et al., Nucleic Acids Res., 18, 6069-6074, 1990 and Rashtchian, A. et al., Biochem., 206, 91-97,1992 where a DNA fragment with single-stranded 5′ and 3′ ends is ligated into a desired vector which can then be amplified in vivo. These methods are useful in making constructs useful in methods of plants of the invention, wherein the gene suppression element of the construct can include at least one anti-sense sequence, and optionally a sense sequence complementary to the at least one anti-sense sequence.

The DNA constructs for enhanced anti-sense transcription units of this invention will simply comprise a promoter element operably linked to an anti-sense oriented DNA. DNA constructs for enhanced anti-sense transcription units can be stacked with recombinant DNA for imparting other traits e.g. herbicide resistance or pest resistance or other trait such as cold germination tolerance, water deficit tolerance and the like, e.g. by expressing or suppressing other genes. Constructs for coordinated decrease and increase of gene expression are disclosed in U.S. patent application Publication 2004/0126845 A1, incorporated herein by reference.

Depending on the application the promoter used to transcribe the anti-sense RNA may be constitutive, tissue specific or inducible. See U.S. Pat. Nos. 5,858,742 and 5,322,938 which disclose versions of the constitutive promoter derived from cauliflower mosaic virus (CaMV35S), U.S. Pat. No. 5,641,876 which discloses a constitutive rice actin promoter, U.S. Pat. No. 6,429,357 which discloses a constitutive rice actin 2 promoter and intron and U.S. Pat. No. 6,177,611 which discloses constitutive maize promoters,. See U.S. Pat. Nos. 5,837,848; 6,437,217 and 6,426,446 which disclose root specific promoters and U.S. Pat. No. 6,433,252 which discloses a maize L3 oleosin promoter. See also U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters and U.S. patent application Publication 2004/0123347 A1 which discloses water deficit inducible promoters. All of the above-described patents disclosing promoters and their use in recombinant DNA constructs in plants are incorporated herein by reference.

In transformation practice DNA is introduced into only a small percentage of target cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Methods and compositions for transforming plants by introducing a recombinant DNA construct into a plant genome in the practice of this invention can include any of the well-known and demonstrated methods. A preferred method of plant transformation is microprojectile bombardment as illustrated in U.S. Pat. No. 5,015,580 (soy), U.S. Pat. No. 5,550,318 (corn), U.S. Pat. No. 5,538,880 (corn), U.S. Pat. No. 6,153,812 (wheat), U.S. Pat. No. 6,160,208 (corn), U.S. Pat. No. 6,288,312 (rice) and U.S. Pat. No. 6,399,861 (corn). Another preferred method of plant transformation is Agrobacterium-mediated transformation as illustrated in U.S. Pat. No. 5,159,135 (cotton), U.S. Pat. No. 5,824,877 (soy), U.S. Pat. No. 5,591,616 (corn) and U.S. Pat. No. 6,384,301 (soy). All of the above-described patents disclosing materials and methods for plant transformation are incorporated herein by reference. See also U.S. patent application Publication 2003/0167537 A1, incorporated herein by reference, for a description of vectors, transformation methods, and production of transformed Arabidopsis thaliana plants where transcription factors are constitutively expressed by a CaMV35S promoter.

Transformation methods to provide plants with stably-integrated enhanced anti-sense gene suppression DNA constructs are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Those cells which are capable of proliferating as callus also are recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. Nos. 6,194,636 and 6,232,526 and U.S. application Ser. No. 09/757,089, which are incorporated herein by reference.

The seeds of transgenic plants can be harvested from fertile transgenic plants and be used to grow progeny generations of transformed plants of this invention including hybrid plants line comprising the recombinant DNA construct expressing an agent for genes suppression.

In addition to direct transformation of a plant with a recombinant DNA construct, transgenic plants can be prepared by crossing a first plant having a recombinant DNA construct with a second plant lacking the construct. For example, recombinant DNA can be introduced into a plant line that is amenable to transformation to produce a transgenic plant which can be crossed with a second plant line to introgress the recombinant DNA into the second plant line.

A transgenic plant with recombinant DNA effecting gene suppression can be crossed with plant line having other recombinant DNA that confers another trait, e.g. yield improvement, herbicide resistance or pest resistance to produce progeny plants having recombinant DNA that confers both gene suppression ant the other trait. Typically, in such breeding for combining traits the transgenic plant donating the additional trait is a male line and the transgenic plant carrying the base traits is the female line. The progeny of this cross will segregate such that some of the plant will carry the DNA for both parental traits and some will carry DNA for one parental trait; such plants can be identified by markers associated with parental recombinant DNA Progeny plants carrying DNA for both parental traits can be crossed back into the female parent line multiple times, e.g. usually 6 to 8 generations, to produce a progeny plant with substantially the same genotype as one original transgenic parental line but for the recombinant DNA of the other transgenic parental line.

### EXAMPLES

### Example 1

This example illustrates the construction and use of vectors designed for double-stranded RNAi suppression or for anti-sense suppression of a luciferase gene. The gene suppression experiments used were similar to a dual luciferase assay described by Horstmann et al. (2004) BMC Biotechnol., 4:13, which is incorporated by reference herein.

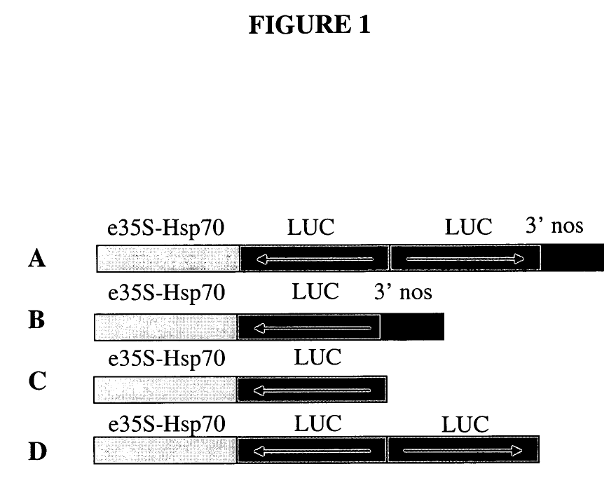

A prior art vector, “vector 1A”, designed for double-stranded RNAi suppression of a luciferase gene was constructed as depicted in FIG. 1A with an RNAi transcription unit with a polyadenylation site including (a) a chimeric promoter including an enhanced CaMV35S promoter linked to an enhancer element (an intron from heat shock protein 70 of Zea mays, Pe35S-Hsp intron), (b) an inverted repeat of DNA coding for firefly luciferase (LUC) with anti-sense oriented DNA followed by a sense oriented DNA, and (c) a 3′UTR DNA from Agrobacterium tumefaciens nopaline synthase gene (3′NOS) which provides a polyadenylation (polyA) site. Elements of the plasmid comprising the RNAi transcription unit had a DNA sequence of SEQ ID NO. 1. See Table 1 for a description of the elements within SEQ ID NO. 1.

TABLE 1

| | Nucleotide position |
|---|---|
| Element | in SEQ ID NO. 1 |
| CaMV e35S promoter | 1-614 |
| Hsp 70 intron | 645-1448 |
| Firefly luciferase anti-sense | 1455-1025 |
| Firefly luciferase sense | 2082-2502 |
| 3′ UTR from nopaline synthase | 2515-2767 |

A prior art vector, “vector 1B”, designed for anti-sense suppression of a luciferase gene and containing a polyA site was constructed as depicted in FIG. 1B with an anti-sense transcription unit including (a) the CaMV e35S - Hsp 70 intron chimeric promoter as described in Table 1, (b) the firefly luciferase anti-sense sequence described in Table 2, and (c) the 3′ UTR from nopaline synthase as described in Table 1.

A novel vector, “vector IC”, designed for double-stranded RNAi suppression of a luciferase gene was constructed as depicted in FIG. 1C with an RNAi transcription unit without a polyadenylation site and including (a) the CaMV e35S - Hsp 70 intron chimeric promoter as described in Table 1, and (b) an inverted repeat of DNA coding for firefly luciferase, including the firefly luciferase anti-sense and firefly luciferase sense sequences described in Table 1. The RNAi transcription unit did not have 3′UTR DNA sequence providing a functional polyadenylation site.

Another novel vector, “vector ID”, designed for anti-sense suppression of a luciferase gene and without a functional polyadenylation site was constructed as depicted in FIG. 1D with an anti-sense transcription unit without polyadenylation site and including (a) the CaMV e35S - Hsp 70 intron chimeric promoter as described in Table 1, and (b) the firefly luciferase anti-sense sequence described in Table 1. The RNAi transcription unit did not have 3′UTR DNA sequence providing a functional polyadenylation site.

Maize protoplasts were prepared as previously described by Sheen (1990) Plant Cell, 2:1027-1038, which is incorporated by reference herein. Each of the four vectors 1A through 1D was electroporated together with reporter vectors for firefly luciferase and Renilla luciferase into three separate volumes of maize protoplasts. Two sets of firefly luciferase suppression experiments were performed to confirm the enhanced ability for gene suppression exhibited by the constructs without a functional polyadenylation site (vectors 1C and 1D) relative to the anti-sense construct with a functional polyadenylation site (vector 1B). The relative level of suppression of the target gene, firefly luciferase, was indicated by the ratio of firefly luciferase to Renilla luciferase “ffLUC/rLUC”, and the results of the two experiments are given in Table 2.

TABLE 2

| | | Average ffLUC/rLUC | |
|---|---|---|---|
| | | First | Second |
| Vector | Description of Construct | experiment | experiment |
| 1A | RNAi with polyA site | 1862 | 2387 |
| 1B | anti-sense with polyA site | 6089 | 13988 |
| 1C | RNAi without polyA site | 3620 | 5879 |
| 1D | anti-sense without polyA site | 2238 | 4762 |

### Example 2

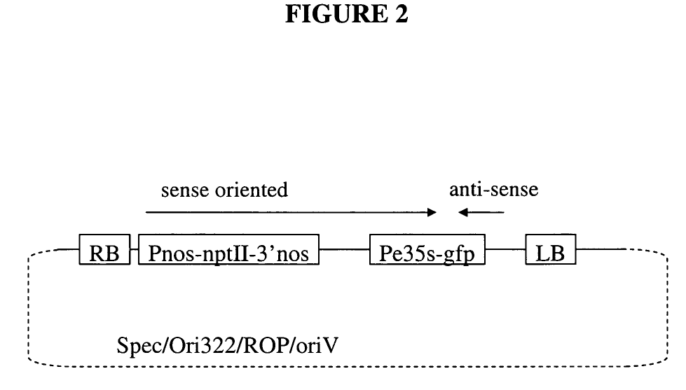

This example describes transformation of a crop plant (maize) with an enhanced anti-sense construct. A plasmid for binary vector Agrobacterium-mediated transformation of maize is constructed including the elements shown in FIG. 2. Specifically, the plasmid includes an nptII gene as an antibiotic selectable marker and a recombinant DNA construct for enhanced anti-sense gene suppression, consisting of a CaMV35S promoter operably linked to transcribable DNA consisting of about 300 base pairs of a green fluorescent protein (gfp) gene in an anti-sense orientation, wherein a functional polyadenylation site is absent in this transcribable DNA. The plasmid also includes left T-DNA border (LB) and right T-DNA border (RB) elements. A control plasmid for RNAi suppression of green fluorescent protein (GFP) is constructed by adding to the enhanced anti-sense construct shown in FIG. 2 a repeat of the gfp DNA in the sense orientation followed by a 3′ NOS element including a functional polyadenylation site. Maize callus for transformation is selected from a transgenic maize line expressing GFP. Both the plasmid with the enhanced anti-sense construct and the control plasmid with the RNAi construct are inserted into maize callus by Agrobacterium-mediated transformation. Events are selected as being resistant to kanamycin. The efficiency of non-systemic suppression with enhanced anti-sense constructs is substantially the same as with the RNAi constructs.

### Example 4

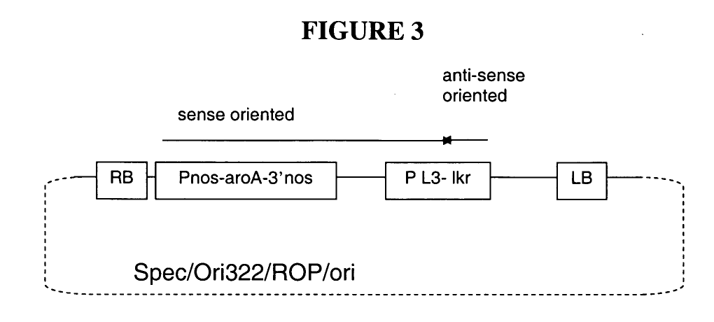

This example illustrates the use of a recombinant DNA construct for non-systemic suppression of a target gene in specific tissue of a transgenic plant. Specifically, this example describes transformation of a crop plant (maize) with an enhanced anti-sense construct. A plasmid for binary vector Agrobacterium-mediated transformation of corn is constructed including the elements shown in FIG. 3. Specifically, the plasmid includes an aroA gene as an herbicidal selectable marker and a recombinant DNA construct for enhanced anti-sense gene suppression, consisting of a seed-specific maize L3 oleosin promoter (as disclosed in U.S. Pat. No. 6,433,252, incorporated herein by reference) operably linked to transcribable DNA consisting of about 300 base pairs of the LKR domain of a maize lysine ketoglutarate reductase/saccharopine dehydrogenase gene (LKR/SDH) in an anti-sense orientation, wherein a functional polyadenylation site is absent in this transcribable DNA. The plasmid also includes left T-DNA border (LB) and right T-DNA border (RB) elements. An alterative vector contains an additional sense DNA sequence that is complementary to the LKR anti-sense sequence, allowing transcription of an at least partially double-stranded RNA from the construct. The plasmid with the enhanced anti-sense construct is inserted into maize callus by Agrobacterium-mediated transformation. Events are selected as being resistance to glyphosate herbicide and grown into transgenic maize plants to produce F1 seed. Mature seeds from each event are analyzed to determine success of transformation and suppression of LKR/SDH. The mature transgenic seeds are dissected to extract protein for Western analysis. Seed from transgenic maize plants shows reduction in LKR/SDH

### Example 5

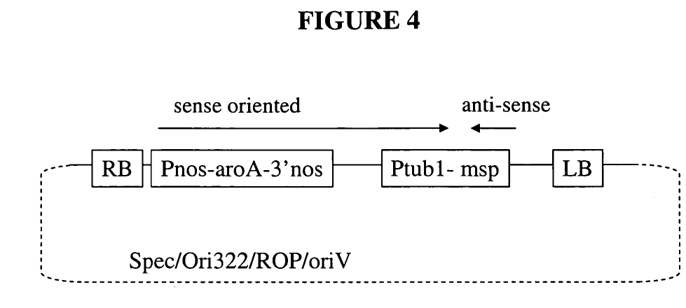

This example illustrates use of recombinant DNA constructs for pest control in plants producing by means of non-systemic gene suppression in a specific tissue of a transgenic plant. Specifically, this example describes transformation of a crop plant (soybean) with an enhanced anti-sense construct. A plasmid for binary vector Agrobacterium-mediated transformation of soybean is constructed including the elements shown in FIG. 4. Specifically, the plasmid includes an aroA gene as an herbicidal selectable marker and a recombinant DNA construct for enhanced anti-sense gene suppression, consisting of a TUB-1 root specific promoter from Arabidopsis thaliana (disclosed in FIG. 1 of U.S. patent application Publication 2004/078841 A1, incorporated by reference herein) operably linked to transcribable DNA consisting of anti-sense oriented DNA of a nematode major sperm protein (msp) of a soybean cyst nematode (disclosed as SEQ ID NO:5 in U.S. patent application Publication 2004/0098761 A1, incorporated herein by reference), wherein a functional polyadenylation site is absent in this transcribable DNA. The plasmid also includes left T-DNA border (LB) and right T-DNA border (RB) elements. An alterative vector contains an additional sense DNA sequence that is complementary to the LKR anti-sense sequence, allowing transcription of an at least partially double-stranded RNA from the construct. The plasmid with the enhanced anti-sense construct is inserted into soybean callus by Agrobacterium-mediated transformation. Events are selected as being resistance to glyphosate herbicide. Reduction in soybean cyst nematode infestation as compared to wild type is observed.

All of the materials and methods disclosed and claimed herein can be made and used without undue experimentation as instructed by the above disclosure. Although the materials and methods of this invention have been described in terms of preferred embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of tissue-specific control of a pest or pathogen of a plant, comprising providing a transgenic plant having in its genome a recombinant DNA construct comprising a tissue-specific promoter operably linked to a gene suppression element, wherein said gene suppression element transcribes to RNA that suppresses at least one gene of said pest or pathogen and is retained in the nucleus, thereby providing tissue-specific control of said pest or pathogen.

2. The method of claim 1, wherein said pest or pathogen is an invertebrate.

3. The method of claim 2, wherein said invertebrate is an insect.

4. The method of claim 2, wherein said invertebrate is a nematode.

5. The method of claim 3, wherein said insect is a coleopteran.

6. The method of claim 4, wherein said nematode is a root nematode and said tissue-specific promoter is a root-specific promoter.

7. A transgenic plant that is resistant to a pest or pathogen of said plant, wherein said transgenic plant has in its genome a recombinant DNA construct comprising a tissue-specific promoter operably linked to a gene suppression element, wherein said gene suppression element transcribes to RNA that suppresses at least one gene of said pest or pathogen and is retained in the nucleus, thereby providing tissue-specific control of said pest or pathogen.

8. The transgenic plant of claim 7, wherein said transgenic plant is a transgenic crop plant and said pest or pathogen is an invertebrate.

9. The transgenic plant of claim 8, wherein said invertebrate is a nematode.

10. The transgenic plant of claim 8, wherein said invertebrate is an insect.

11. A method of non-systemic suppression of at least one target gene, comprising transcribing in a transgenic plant a recombinant DNA construct consisting of a promoter operably linked to a gene suppression element, wherein transcription of said gene suppression element produces RNA that non-systemically suppresses at least one target gene and is retained in the nucleus, thereby suppressing said target gene relative to expression in the absence of said transcription.

12. The method of claim 11, wherein said at least one target gene is selected from the group consisting of a gene native to said transgenic plant, a transgene in said transgenic plant, a gene native to a pest or pathogen of said transgenic plant, and a microRNA precursor DNA sequence.

13. The method of claim 11, wherein said at least one target gene is multiple target genes.

14. The method of claim 11, wherein said promoter is a tissue-specific promoter or an inducible promoter.

15. The method of claim 11, wherein said promoter is a seed-specific promoter and said non-systemic suppression is seed-specific suppression of said at least one target gene.
